(19) United States
(12) Patent Application Publication
(10) Pub. No.: US 2016/0304584 A1
(43) Pub. Date: Oct. 20, 2016

(54) MAFB MUTANTS AND USES THEREOF

(71) Applicants: INSERM (INSTITUT NATIONAL DE LA SANTÉ ET DE LA RECHERCHE MÉDICALE, Paris (FR); EUROPEAN MOLECULAR BIOLOGY LABORATORY, Heidelberg (DE); CENTRE NATIONAL DE LA RECHERCHE SCIENTIFIQUE (CNRS), Paris (FR); UNIVERSITÉ D'AIX MARSEILLE, Marseille Cedex 07 (FR)

(72) Inventors: Michael SIEWEKE, Marseille (FR); Vivian POGENBERG, Hamburg (DE); Matthias WILMANNS, Hamburg (DE); Larissa CONSANI TEXTOR, Hamburg (DE); Laurent VANHILLE, Marseille (FR)

(21) Appl. No.: 15/101,425

(22) PCT Filed: Dec. 3, 2014
(86) PCT No.: PCT/EP2014/076365

§ 371 Date: Jun. 3, 2016

(30) Foreign Application Priority Data
Dec. 3, 2013 (EP) 13306661.3

(51) Int. Cl. C07K 14/82; G01N 33/50; C07K 16/32

(52) U.S. Cl. CPC C07K 14/82; C07K 16/32; G01N 33/502; C07K 2319/73; C07K 2319/80

(57) ABSTRACT

The present invention relates to MafB mutants and uses thereof for research, screening and therapeutic purposes. In particular, the present invention relates to a MafB mutant polypeptide comprising the sequence as set forth in SEQ ID NO: 1 or 2 or a function conservative variant thereof wherein the glutaminic acid residue at position 269 has been deleted or substituted with a basic amino acid. The present invention also relates to a MafB mutant polypeptide comprising the sequence as set forth in SEQ ID NO: 1 or 2 or a function conservative variant thereof wherein the valine residue at position 277 has been deleted or substituted with a polar amino acid.

### FIELD OF THE INVENTION

The present invention relates to MafB mutants and uses thereof for research, screening and therapeutic purposes.

### BACKGROUND OF THE INVENTION

Tissue macrophages serve important roles in the immune response, tissue homeostasis, metabolism and repair. Because of these multifaceted activities, macrophages have been identified as key players (and targets) in diseases with major importance for public health, such as cancer, cardiovascular, autoimmune, chronic inflammatory, degenerative and metabolic diseases. In macrophages MafB is a transcription factor that is constitutively expressed and it was postulated that MafB represents a key element for modulating the expression of genes, which control the inflammatory or immunosuppressive phenotype of macrophages. In addition, the transcription factor c-Fos is transiently induced upon pathogen challenge or cytokine stimulation and has functions in modulating macrophage activation (Amit et al., 2009; Introna et al., 1986; Koga et al., 2009; Pulendran et al., 2010). In terms of overall domain structure, MafB is a member of the Maf sub-family of bZip transcription factors, which share an additional helical bundle region known as the extended homology region (EHR) preceding the BR (Kerppola and Curran, 1994; Kusunoki et al., 2002). MafB requires a long DNA-recognition sequence known as the Maf-recognition element (MARE) that includes a three-base extension of the central seven or eight base pair (bp) CRE/TRE core motif of the canonical bZip recognition element (Yamamoto et al., 2006).

### SUMMARY OF THE INVENTION

The present invention relates to MafB mutants and uses thereof for research, screening and therapeutic purposes. In particular, the present invention is defined by the claims.

### DETAILED DESCRIPTION OF THE INVENTION

The inventors determined two high-resolution structures of the transcription factor MafB as a homodimer and as a heterodimer with c-Fos bound to variants of the Maf-recognition element. The structures revealed several unexpected and dimer-specific coiled coil-heptad interactions. Based on these findings, they have engineered two MafB mutants with opposite dimerization preferences. One of them showed a strong preference for MafB/c-Fos heterodimerization and enabled selection of heterodimer-favoring over homodimer-specific Maf-recognition element variants. Taken together, this study shows that, by making use of structural leucine zipper interaction data, it is possible to alter the balance of homo- and heterodimeric MafB complexes in the presence of the same T-MARE sequence and the selection of hetero-dimer favoring DNA binding sites over homo-dimer favoring DNA binding sites by introducing additional targeted attractive or repulsive interactions. Engineered MafB dimerization may therefore ultimately enable the precise understanding and controlled manipulation of distinct activity states and binding repertoires of MafB, in particular in macrophages, with therapeutic potential in infectious disease, inflammatory or autoimmune disorders, regeneration and tumor biology. Accordingly, the present invention intends to protect these new MafB mutant polypeptides and their uses for research, screening and therapeutic purposes.

As used herein the term “MafB” has its general meaning in the art and refers to the MafB transcription factor. This gene is expressed in a variety of cell types (including lens epithelial, pancreas endocrine, chondrocyte, neuronal and hematopoietic cells) and encodes a protein of 323 amino acids containing a typical bZip motif in its carboxy-terminal region. MafB can form a homodimer and specifically binds Maf-recognition elements (MAREs) palindromes, composite AP-1/MARE sites or MARE halfsites with AT rich 5′ extensions (Yoshida, et al. 2005). In addition, MafB can form heterodimers with c-Fos. Exemplary amino acid sequences of MafB are represented by SEQ ID NO:1 or SEQ ID NO:2.

| SEQ ID NO: 1 MafB (Homo Sapiens): | |
|---|---|
| 1 | maaelsmgpe lptsplamey vndfdllkfd vkkeplgrae rpgrpctrlq pagsysstpl |
| 61 | tpcssvpss psfspteqkt hledlywmas nyqqmnpeal nltpedavea ligshpvpqp |
| 121 | lqsfdsfrga hhhhhhhhph phhaypgagv andelgphah phhhhhhqas pppssaaspa |
| 181 | qqlptshpgp gphatasata aggngsvedr fsddqlvsms vrelnrhlrg ftkdevirlk |
| 241 | qkrrtlknrg yaqscrykrv qqkhhlenek tqliqqveql kqevsrlare rdaykvkcek |
| 301 | lansgfreag stsdspsspe ffl |
| SEQ ID NO: 2: MafB (Mus Musculus) | |
| 1 | maaelsmgqe lptsplamey vndfdllkfd vkkeplgrae rpgrpctrlq pagsysstpl |
| 61 | stpcssvpss psfsptepkt hledlywmas nyqqmnpeal nltpedavea ligshpvpqp |
| 121 | lqsfdgfrsa hhhhhhhhph phhgypgagv thddlgqhah phhhhhhqas pppssaaspa |
| 181 | qqlptshpgp gphataaata aggngsvedr fsddqlvsms vrelnrhlrg ftkdevirlk |
| 241 | qkrrtlknrg yaqscrykrv qqkhhlenek tqliqqveql kqevsrlare rdaykvkcek |
| 301 | lansgfreag stsdspsspe ffl |

The present invention relates to a MafB mutant polypeptide comprising the sequence as set forth in SEQ ID NO: 1 or 2 or a function conservative variant thereof wherein the glutaminic acid residue at position 269 has been deleted or substituted with a basic amino acid.

Typically, the basic amino acid is selected from the group consisting of arginine, lysine or histidine

The present invention relates to a MafB mutant polypeptide comprising the sequence as set forth in SEQ ID NO: 1 or 2 or a function conservative variant thereof wherein the valine residue at position 277 has been deleted or substituted with a polar amino acid.

Typically, the polar amino acid is selected from the group consisting of serine (Ser), threonine (Thr), cysteine (Cys), asparagine (Asn), glutamine (Gln), and tyrosine (Tyr).

The polypeptides of the invention may be produced by any technique known per se in the art, such as, without limitation, any chemical, biological, genetic or enzymatic technique, either alone or in combination.

A further object of the present invention encompasses function-conservative variants of the polypeptides of the present invention, providing that the glutaminic acid residue at position 269 or the valine residue at position 277 remains deleted or substituted as described above.

“Function-conservative variants” are those in which a given amino acid residue in a protein has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity.

Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, most preferably at least 85%, and even more preferably at least 90%, and which has the same or substantially similar properties or functions as the native or parent protein to which it is compared.

In some embodiments, the polypeptide of the invention consists or comprises a sequence having at least 90% amino acid identity with SEQ ID NO: 1 or 2 providing that the glutaminic acid residue at position 269 or the valine residue at position 277 remains deleted or substituted as described above. In some embodiments, the polypeptide of the invention consists or comprises a sequence having 90; 91; 92; 93; 94; 95; 96; 97; 98 or 99% amino acid identity with SEQ ID NO: 1 or 2 providing that the glutaminic acid residue at position 269 or the valine residue at position 277 remains deleted or substituted as described above.

A further aspect of the invention relates to a fusion protein comprising a polypeptide as above described fused to a heterologous polypeptide (i.e. a polypeptide that do not derive from a polypeptide of the invention).

As used herein, a fusion protein” comprises all or part (typically biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of the polypeptide of the invention.

One useful fusion protein is a GST fusion protein in which the polypeptide of the invention is fused to the C-terminus of GST sequences. Such fusion proteins can facilitate the purification of a recombinant polypeptide of the invention.

In some embodiments, the fusion protein contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In some embodiments, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain. Even more the signal sequence can represent a sequence that will facilitate the production of the protein of interest in particular cell interest. For example, the polypeptide of the invention may be fused to a sequence that will drive the expression of the polypeptide in the exosomes or microparticles (or other vesicles)

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus. Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

The polypeptides of the invention can exhibit post-translational modifications, including, but not limited to glycosylations, (e.g., N-linked or O-linked glycosylations), myristylations, palmitylations, acetylations and phosphorylations (e.g., serine/threonine or tyrosine).

The polypeptides of the invention may be produced by any technique known per se in the art, such as, without limitation, any chemical, biological, genetic or enzymatic technique, either alone or in combination. For example, knowing the amino acid sequence of the desired sequence, one skilled in the art can readily produce said polypeptides, by standard techniques for production of polypeptides. For instance, they can be synthesized using well-known solid phase method, typically using a commercially available peptide synthesis apparatus. Alternatively, the polypeptides of the invention can be synthesized by recombinant DNA techniques as is now well-known in the art. For example, the polypeptides can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques.

One aspect of the invention pertains to isolated nucleic acid molecules that encode a polypeptide of the invention.

A nucleic acid molecule of the present invention can be isolated using standard molecular biology techniques and the sequence information provided herein. Using all or a portion of the nucleic acid sequences of the invention as a hybridization probe, nucleic acid molecules of the invention can be isolated using standard hybridization and cloning techniques.

A nucleic acid molecule of the invention can be amplified using cDNA, mRNA or genomic DNA as a template and appropriate oligonucleotide primers according to standard methods. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

The skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologues of various species may be non-essential for activity and thus would be likely targets for alteration. Alternatively, amino acid residues that are conserved among the homologues of various species (e.g., mouse and human) may be essential for activity and thus would not be likely targets for alteration.

Mutations can be introduced by standard techniques, such as site-directed mutagenesis and PCR-mediated mutagenesis. Preferably, conservative amino acid substitutions are made at one or more predicted non-essential amino acid residues.

Following mutagenesis, the encoded protein can be expressed recombinantly and the activity of the protein can be determined.

Typically, said nucleic acid is a DNA or RNA molecule, which may be included in any suitable vector, such as a plasmid, cosmid, episome, artificial chromosome, phage or a viral vector. The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce polypeptides of the invention, fragments thereof or fusion proteins according to the invention.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA synthesis techniques.

In some embodiments, the expression vector is a yeast expression vector.

Alternatively, the expression vector is a baculovirus expression vector.

In yet another embodiment, a nucleic acid of the invention is expressed in mammalian cells using a mammalian expression vector. When used in mammalian cells, the expression vector's control functions are often provided by viral regulatory elements. For example, commonly used promoters are derived from polyoma, Adenovirus 2, cytomegalovirus and Simian Virus 40.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art.

Another aspect of the invention pertains to host cells into which a recombinant expression vector of the invention has been introduced.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

In some embodiments, the host cell is a monocyte or a macrophage, and more particularly a self renewal monocyte or macrophage. Typically, a self renewing monocyte or macrophage is a Maf DKO monocyte or macrophage (c-Maf −/−, Mafb −/−) as described in Aziz A, Soucie E, Sarrazin S, Sieweke M H. MafB/c-Maf deficiency enables self-renewal of differentiated functional macrophages. Science. 2009 Nov. 6; 326(5954):867-71. doi: 10.1126/science.1176056.

Vector DNA can be introduced into prokaryotic or eukaryotic cells (e.g. macrophage) via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In some embodiments, the expression characteristics of an endogenous gene within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene and controls, modulates or activates. For example, endogenous genes which are normally “transcriptionally silent”, i.e., genes which are normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous genes may be activated by insertion of a promiscuous regulatory element that works across cell types. A heterologous regulatory element may be inserted into a stable cell line or cloned microorganism, such that it is operatively linked with and activates expression of endogenous genes, using techniques, such as targeted homologous recombination, which are well known to those of skill in the art.

A host cell of the invention, such as a prokaryotic or eukaryotic host cell in culture, can be used to produce a polypeptide of the invention. Accordingly, the invention further provides methods for producing a polypeptide of the invention using the host cells of the invention. In some embodiments, the method comprises culturing the host cell of invention (into which a recombinant expression vector encoding the polypeptide has been introduced) in a suitable medium such that the polypeptide is produced. In some embodiments, the method further comprises isolating the polypeptide from the medium or the host cell.

The present invention also relates to a method for producing a recombinant host cell expressing an polypeptide according to the invention, said method comprising the steps consisting of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said polypeptide. Such recombinant host cells can be used for the production of polypeptides according to the present invention, as previously described.

The invention further relates to a method of producing a polypeptide according to the invention, which method comprises the steps consisting of: (i) culturing a transformed host cell according to the invention under conditions suitable to allow expression of said polypeptide; and (ii) recovering the expressed polypeptide.

The host cells of the invention can also be used to produce non human transgenic animals. For example, in some embodiments, a host cell of the invention is a fertilized oocyte or an embryonic stem cell into which a sequence encoding a polypeptide of the invention has been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity.

As used herein, a “transgenic animal” is a non-human animal, typically a mammal, more typically a rodent such as a rat or mouse, in which one or more of the cells of the animal includes a transgene. Examples of transgenic animals include rodents such as mouse or rat, non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal.

A transgenic animal of the invention can be created by introducing nucleic acid encoding a polypeptide of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

In some embodiments, transgenic non-human animals can be produced which contain selected systems which allow for regulated expression of the transgene. One example of such a system is the cre/toxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

The present invention also relates to an antibody specific for a polypeptide of the invention. In particular, the antibodies of the invention specifically recognize the polypeptide of the invention (i.e. a MafB mutant polypeptide of the invention) over non mutant (wild type) polypeptide.

In particular, monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of polypeptide of the invention (or a fragment thereof). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Polypeptides, nucleic acids, vector, host cells, and animal model are particularly suitable for research, screening and therapeutic purposes.

In particular, the host cells and animal models may be used for analysing gene expression of said cells (e.g. by Chip-seq analysing) or in said animals, and determining what are and how the gene(s) could contribute to a phenotype (e.g. invalidating the expression of a gene or of a plurality of genes).

The host cells and animal models of the invention could be particularly suitable for in vitro and in vivo analysing particular phenotypes, especially, in particular conditions that can mimic a particular physiological (e.g. physiopathological) context.

The host cells of the invention in particular monocytes or macrophages of the invention may also be screened for the expression at their surface of specific markers (for example expression of CD molecules, receptors . . . ). Said surface markers will be thus representative of certain phenotypes that can be retrieved in some physiopathological conditions and thus could represent biomarkers of interest for diagnostic purposes or even could represent specific therapeutic targets that can be then address with monoclonal antibodies (e.g. chimeric, humanized or full human monoclonal antibodies), polypeptides or small organic compounds for therapeutic purposes. Comparison between host cells favouring the formation of the homodimeric complexes and host cells favouring the formation of the heterodimeric complexes will be thus particularly suitable for indentified said specific surface markers. Moreover the cells may be placed under conditions mimicking physiopathological conditions for screening specific surface markers of interest.

The host cells and animal models of the invention could also be particularly suitable of screening a drug. Typically, said drug may be an inhibitor of the activity of the homodimer complex (i.e. MafB/MafB complex), of the heterodimer complex (i.e. c-Fos/MafB complex) or of both complexes. Typically, said drugs can inhibit or reduce the transcription from promoters containing binding sites for the complexes. For example the compound may block the interaction of the complex with the binding sequences specific for the complex, or may bind to the complex in manner that the complex is not able to bind to its binding sites.

Thus the present invention also relates to a method for screening a plurality of candidate compounds comprising the steps consisting of (a) testing each of the candidate compounds for its ability to inhibit the activity of the homodimeric or heterodimeric complex and (b) and positively selecting the candidate compounds capable of inhibiting said activity.

Typically, the candidate compound is selected from the group consisting of small organic molecules, peptides, polypeptides or oligonucleotides (siRNAs, or antisense oligonucleotides).

Testing whether a candidate compound can inhibit the activity of the complex can be determined using or routinely modifying reporter assays known in the art. For example, the method may involve contacting the appropriate host cell of the invention with the candidate compound, and measuring the mediated transcription by the complex (e.g., activation of promoters containing the binding sites for the complex), and comparing the cellular response to a standard cellular response. Typically, the standard cellular response is measured in absence of the candidate compound. A decrease cellular response over the standard indicates that the candidate compound is an inhibitor of the activity of the complex. In another embodiment the invention provides a method for identifying a ligand which binds specifically to the complex. The method may also involve screening for compounds which inhibit the activity of the complex by determining, for example, the amount of transcription from promoters containing the binding sites for the complex in the appropriate host cell of the invention. Such a method may involve contacting the appropriate host cell of the invention with a candidate compound, and determining the amount of transcription from promoters containing the binding sites for the complex. A reporter gene (e.g, GFP) linked to a promoter containing an the binding sites may be used in such a method, in which case, the amount of transcription from the reporter gene may be measured by assaying the level of reporter gene product, or the level of activity of the reporter gene product in the case where the reporter gene is an enzyme. A decrease in the amount of transcription from promoters containing the binding sites in the host cell, compared to a host cell not contacted with the candidate compound, would indicate that the candidate compound is an inhibitor of the activity of the complex.

The candidate compounds that have been positively selected may be subjected to further selection steps in view of further assaying its in vitro properties on cells or interests such as monocytes or macrophages. For example, the candidate compounds that have been positively selected with the screening method as above described may be further selected for their ability to inhibit or enhance the migration and/or proliferation of the cells of interest (e.g. monocytes and/or macrophages) or for their ability to inhibit or enhance of expression of certain specific factors (e.g. cytokines, interleukins), typically factors associated with inflammation or immunosupression. The in vitro methods as above described may be performed by adding an amount of the candidate compound to be tested to the culture medium of the cells of interests. Usually, a plurality of culture samples are prepared, so as to add increasing amounts of the candidate compound to be tested in distinct culture samples. Generally, at least one culture sample without candidate compound is also prepared as a negative control for further comparison. Finally, the candidate compounds that have been positively selected may be subjected to further selection steps in view of further assaying its in vivo properties on animal models (e.g. animal models representative of a physiopathological condition such as an inflammatory condition). Typically, the positively selected candidate compound may be administered to the animal model and the progression of the condition is determined and compared with the progression of condition in an animal model that was not administered with the candidate compound.

In particular, the identified compounds that are able to inhibit or reduce an inflammatory phenotype or even that are able to favour an immunosuppressive phenotype may particularly suitable for the treatment of an undesirable inflammatory condition in a subject in need thereof, or may be suitable for the treatment of auto-immune diseases or cancers.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy. For example, treating an undesirable inflammatory condition, within the context of the invention, refers to inflammation that includes clinically detrimental macrophage activation as a component.

Administering the drugs identified by the screening methods of the invention may be useful to reduce undesirable inflammation conditions in any number of pathologies, including, but not limited to, colitis, alveolitis, bronchiolitis obliterans, ileitis, pancreatitis, glomerulonephritis, uveitis, arthritis, hepatitis, dermatitis, and enteritis, acute and chronic conditions in cardiovascular, e.g., acute myocardial infarction; central nervous system injury, e.g., stroke, traumatic brain injury, spinal cord injury; peripheral vascular disease; pulmonary, e.g., asthma, ARDS; autoimmune, e.g., rheumatoid arthritis, multiple sclerosis, lupus, sclerodoma; psoriasis; gastrointestinal, e.g., graft-versus-host-disease, Crohn's disease, diabetes, ulcerative colitis, acute and chronic transplantation rejection, dermatitis, colitis, alveolitis, bronchiolitis obliterans, ileitis, pancreatitis, glomerulonephritis, uveitis, arthritis, hepatitis, dermatitis, and enteritis. Administering the drugs may also be useful to reduce undesirable inflammation conditions in any number of CNS pathologies, including, but not limited to, ischemic stroke, multiple sclerosis, Alzheimer's Disease, ALS, Parkinson's Disease, hypoxic-ischemia, neonatal hypoxic ischemia, and traumatic brain or spinal cord injury.

The present invention also provides a pharmaceutical composition comprising a host cell of the invention (e.g. monocytes or macrophages), in combination with a pharmaceutically acceptable carrier.

The mononuclear phagocyte system (monocytes and macrophages) represents a distributed organ responsible for homeostasis within the host. Said system is involved in every disease process in which there is persistent tissue injury or metabolic disturbance. Macrophages and monocytes mediate acute as well as chronic inflammation, and promote repair through removal of dead cells and fibrin by phagocytosis and fibrinolysis, induce blood vessel ingrowth (angiogenesis) and modulate fibroblast invasion and production of extracellular matrix. They produce mediators that mobilize systemic responses of the host including fever, release and catabolize stress and other hormones, increase metabolic activity of other cells, and influence blood flow to tissues and capillary permeability. The macrophages themselves display considerable heterogeneity in their functions, often expressing activators as well as inhibitors of a property, e.g. proteolytic activity, or pro- and anti-inflammatory cytokine production, depending on the evolution of a particular host response.

Thus a further object of the invention relates to a method for the treatment of a disease selected from the group consisting of a cancer, acute or acquired immuno-deficiencies, chronic or acute injury, wounds, degenerative diseases, autoimmune diseases, chronic inflammatory diseases, atherosclerosis, poly- and osteo-arthritis, osteoporosis, infectious diseases (e.g. infections by virus, or bacteria), and metabolic diseases in a subject in need thereof comprising administering the subject with a therapeutically effective amount of a population of monocytes and/or macrophages of the invention.

In particular, the invention also relates to a method for treating an undesirable inflammatory condition in a subject in need thereof comprising administering the subject with a therapeutically effective amount of a population of monocytes and/or macrophages expression a recombinant MafB mutant polypeptide comprising the sequence as set forth in SEQ ID NO: 1 or 2 or a function conservative variant thereof wherein the glutaminic acid residue at position 269 has been deleted or substituted with a basic amino acid selected from the group consisting of arginine, lysine or histidine.

In a particular the monocytes and/or macrophages that are administered to the subject are self-renewal monocytes or macrophages as above described.

By a “therapeutically effective amount” of a cell as above described is meant a sufficient amount of said cell to treat a disease or disorder at a reasonable benefit/risk ratio applicable to any medical treatment. It will be understood, however, that the total daily usage of compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific cells employed; and like factors well known in the medical arts

Pharmaceutically acceptable carrier or excipient includes but is not limited to saline, buffered saline, dextrose, water, glycerol and combinations thereof. The carrier and composition can be sterile. The formulation should suit the mode of administration. The composition, if desired, can also contain minor amounts of wetting or emulsifying agents, or pH buffering agents. The composition can be a liquid solution, suspension, or emulsion. In a preferred embodiment, the composition is formulated in accordance with routine procedures as a pharmaceutical composition adapted for intravenous administration to human beings. Typically, compositions for intravenous, administration are solutions in sterile isotonic aqueous buffer. Where necessary, the composition may also include a solubilizing agent and a local anesthetic such as lignocaine to ease pain at the site of the injection.

The invention will be further illustrated by the following figures and examples. However, these examples and figures should not be interpreted in any way as limiting the scope of the present invention.

### Example 1

### Introduction

The transcription of genes is a highly regulated combinatorial process that is mediated by large and dynamic multi-component protein assemblies such as the enhanceosome (Ogata et al., 2003; Panne, 2008; Remenyi et al., 2004). These assemblies generally involve several distinct transcription factors and enhancers that act as homo- or hetero-oligomeric assemblies on specific cis-regulatory DNA promoter and enhancer sites, ultimately modifying the local chromatin architecture to allow the polymerase II machinery to reach the target promoter site. Their ability to assemble with several alternative binding partners allows many transcription factors to be involved in the combinatorial transcription of different genes, leading to unrelated, sometimes even antagonistic, functional readouts or distinct cell fate decisions (Sieweke and Graf, 1998). Understanding the basic mechanisms of variable protein assembly in gene transcription is essential for the rationalization of genotypic and phenotypic effects during development and pathology, and this knowledge of the underlying molecular parameters could ultimately be used to engineer altered transcription circuits.

An important example of such alternative transcription factor assemblies is the basic leucine zipper (bZip) transcription factor family, which bind to a variety of related cyclic AMP-response and 12-O-tetradecanoate 13-acetate (TPA)-response elements (CRE/TRE) DNA-recognition sites both as homo- or heterodimeric complexes (Miller, 2009). Their large combinatorial versatility is established by sequence-specific coiled coil assemblies within the long leucine zipper (Zip) segment, which is next to the basic region (BR) that functions as a direct DNA-binding segment. The molecular determinants of coiled-coil interactions in bZip transcription factors have been extensively studied by biophysical, structural and computational approaches and engineering experiments (Grigoryan and Keating, 2008; Miller, 2009; Vinson et al., 2006). Available data, however, are limited to studies of bZip factors in the absence of DNA and using dimer pairs with similar DNA-binding preferences, such as members of the c-Jun/c-Fos family that have a common preference for CRE/TRE. A direct comparison of alternative bZip complexes with assembly-specific preferences for distinct DNA-recognition elements has not been performed and could identify the determinants of binding repertoire and target gene selection (Miller, 2009; Yamamoto et al., 2006).

To investigate bZip assembly-dependent DNA-binding site selection we have studied two prototype bZip transcription factors with different DNA-binding profiles: MafB and c-Fos. MafB plays important roles in tumorigenesis, differentiation and several developmental processes such as hematopoiesis (Aziz et al., 2009; Eychene et al., 2008; Sarrazin et al., 2009). In particular in macrophages, MafB is constitutively expressed, whereas c-Fos is transiently induced upon pathogen challenge or cytokine stimulation and functions in modulating macrophage activation (Amit et al., 2009; Introna et al., 1986; Koga et al., 2009; Pulendran et al., 2010). In terms of overall domain structure, MafB is a member of the Maf sub-family of bZip transcription factors, which share an additional helical bundle region known as the extended homology region (EHR) preceding the BR (Kerppola and Curran, 1994; Kusunoki et al., 2002, Kataoka K, Noda M, Nishizawa M (1994) Maf nuclear oncoprotein recognizes sequences related to an AP-1 site and forms heterodimers with both Fos and Jun. Mol Cell Biol 14:700-712.). MafB requires a long DNA-recognition sequence known as the Maf-recognition element (MARE) that includes a three-base extension of the central seven or eight base pair (bp) CRE/TRE core motif of the canonical bZip recognition element (Yamamoto et al., 2006). Here we have focused on MARE sequences with a 7-bp TRE-type core (T-MARE). Interestingly, MAREs that have been found in confirmed target genes are highly degenerate both in the TRE core motif and the two flanking regions (Yamamoto et al., 2006). The major reported heterodimeric bZip binding partner of MafB is c-Fos (Kataoka K, Noda M, Nishizawa M (1994) Maf nuclear oncoprotein recognizes sequences related to an AP-1 site and forms heterodimers with both Fos and Jun. Mol Cell Biol 14:700-712.; Newman and Keating, 2003). In contrast to MafB, c-Fos does not homodimerize and its specific DNA interactions are limited to the central CRE/TRE (Glover and Harrison, 1995), thus the presence of the flanking segments of the MARE are not required.

Experimental Procedures:

MafB₂/T-MARE and MafB/c-Fos/T-MARE(Beta) Purification and Crystallization:

The C-terminal region of MafB from Mus musculus (residues 211-305, C298S) was purified as previously described (Textor et al., 2007). Homodimeric MafB₂ complexes were dialyzed overnight into 30 mM Tris-HCl (pH 7.3), 80 mM NaCl, 50 mM MgCl₂ and 3 mM β-mercaptoethanol at room temperature in the presence of an oligonucleotide encompassing the T-MARE-binding site (forward: TAATTGCTGACTCAGCAATT (SEQ ID NO:3); reverse: TAATTGCTGAGTCAGCAATT (SEQ ID NO:4), METABION). The protein-DNA complex was further purified by size exclusion chromatography on a Superdex-75 (16/60) column (GE), equilibrated with the dialysis buffer. The pooled peak fractions corresponding to the (MafB)₂/T-MARE complex were analyzed by SDS-PAGE and native PAGE gels and concentrated to 5 mg/mL, using a centrifugal concentrator with a polyethersulfone membrane with a 10,000 MWCO (VIVASPIN, Sartorius Stedim Biotech). Crystallization drops of 400 nL volume, using a 1:1 protein/mother liquor ratio, were set up in 96-well sitting-drop plates and allowed to equilibrate at 19° C. Diffracting crystals grew in the presence of 0.1 M Na-citrate (pH 5.0), 15% [w/v] PEG-4000, 5% [v/v] PEG-400, and 0.1 M MgCl₂.

For structural investigations of heterodimeric MafB/c-Fos complexes, a c-Fos fragment from Mus musculus (residues 138-200), encompassing the BR and Zip segment, was cloned in the pET-M11 vector and expressed in the E. coli strain BL21(DE3)RIL. Cell pellets with over-expressed MafB and c-Fos were lysed separately under denaturing conditions in 4M urea, 20 mM Tris-HCl (pH7.5), 150 mM NaCl and 15 mM beta-mercaptoethanol. The proteins were separately purified on a NiNTA affinity column (Qiagen). The heterodimer was refolded upon dialysis against a buffer containing 150 mM NaCl, 15 mM Tris pH 7.5, and 10 mM DTT, in the presence of the chemically synthesized cognate DNA duplex T-MARE(beta) (METABION) and the TEV protease in a mass ratio of 1:25 to remove the hexa-histidine tag. The MafB/c-Fos/T-MARE(beta) complex was further purified by exclusion chromatography on a Superdex 75 column, equilibrated with the same buffer. Finally, the MafB/c-Fos/T-MARE(beta) complex was concentrated up to 15 mg/mL, using a centrifugal concentrator with a polyethersulfone membrane with a 10,000 MWCO (VIVASPIN, Sartorius Stedim Biotech). Hanging drop crystallization trials were carried out at 20° C., by mixing equal volumes of reservoir solution and MafB/c-Fos/T-MARE(β) complex solution. Crystals grew from 0.075 M HEPES-Na (pH 7.5), 0.6 M sodium dihydrogen phosphate, 0.6 M potassium dihydrogen phosphate, 25% (v/v) glycerol.

MafB mutants were generated with the QuikChange® Site-DirectedMutagenesis Kit (Stratagene). Primers of 45 base pairs were designed to introduce single mutations in MafB. MafB mutants were expressed and purified as the wild-type protein.

X-ray Structure Determination:

MafB₂/T-MARE. Crystals were cryo-protected by briefly soaking them into a solution used for crystallization, which included in addition 20% [w/v] PEG-400. A native X-ray data set was collected to a resolution of 2.9 Å on beamline ID23-2 at ESRF, Grenoble, France. The data were processed in the orthorhombic Laue group P222 with the XDS package (Kabsch, 2010). The structure was solved by molecular replacement in space group P22121 with the program PHASER (McCoy, 2007), applying the coordinates of one MafB protomer (residues 212-253) bound to the C-MARE half-site from the MafB₂/C-MARE complex (PDB entry 4auw), as the search model. The remaining protein residues and the DNA bases were built manually using the program COOT (Emsley and Cowtan, 2004) and successive cycles of restrained refinement with the program REFMAC5 (Murshudov et al., 1997). One cycle of simulated annealing was applied to the built model using the program PHENIX (Adams et al., 2011). The atomic coordinates were further refined to a final R_{work} and R_{free} of 23.5% and 27.5%, respectively, using an NCS restraints (Table I). Ordered solvent molecules were added to the protein model, using the program ARP/wARP (Laskowski et al., 1998). The quality of the homodimeric MafB structure was validated using the program PROCHECK (Laskowski et al., 1993).

MafB/c-Fos/T-MARE(beta). All crystals used for X-ray data collection were mounted from the mother liquor onto a cryo-loop (Hampton Research) and directly flash-cooled under the nitrogen beam at 100K. X-ray data were collected on the synchrotron radiation beamlines BW7A and X11 at the DORIS III ring at EMBL/DESY, Hamburg, Germany. Experimental phases were determined from an X-ray data set to a maximum resolution of 3.2 Å, using the single-wavelength anomalous dispersion technique. For this purpose, crystals were derivatized with iodine using the vaporizing-iodine-labeling technique (Miyatake et al., 2006), by placing a small drop of 0.67 M KI/0.47M I2 solution next to the crystallization drop for six hours. The protein was weakly derivatized by this process, which together with the anomalous scattering from the DNA phosphate backbone provided sufficient phasing information by the identification of a total of 24 heavy atom sites to solve the structure using a combination of SHELX and SHARP programs (Bricogne et al., 2003; Schneider and Sheldrick, 2002). In addition, a native dataset of the MafB/c-Fos/T-MARE(beta) complex was collected to a resolution of 2.1 Å. The data set were processed with MOSFLM (Leslie and Powell, 2007), and programs of the CCP4 suite (Collaborative Computational Project Number 4, 1994).

Phase-extension was used to combine the experimentally determined phases with a high-resolution (2.3 Å) native dataset. The final model was built with COOT (Emsley and Cowtan, 2004) and refined with the program REFMAC5 (Murshudov et al., 1997) to a final R_{work} and R_{free} of 22.8% and 25.6%, respectively. The asymmetric unit contains one MafB/c-Fos/DNA complex. The stereochemical quality of the model was assessed by use of the program PROCHEK (Laskowski et al., 1993).

In the structure of the MafB₂/T-MARE complex, all DNA bases and the complete polypeptide chains, except one terminal residue of one of the two MafB molecules (F211) are visible. In the structure of the MafB/c-Fos/T-MARE(beta) complex, the complete T-MARE(beta) motif as well as the complete c-Fos and MafB polypeptide chains are visible, except tree residues from the N-terminus (F211-D213) and two residues from the C-terminus (S304-G305) of the expressed MafB fragment.

Electrophoretic Mobility Shift Assay:

Double-stranded synthetic oligonucleotides corresponding to the T-MARE(a) (TCGTCCCTTATGCTGACTCAGCAATTCTC) (SEQ ID NO:5), to T-MARE(beta) (TCGTCCCCTCCTATGAGTCAGCAATTCTC) (SEQ ID NO:6) or to T-MARE(y) (TCGTCCCTTATGCCTACTAGGCAATTCTC) (SEQ ID NO:7) sites were incubated with Klenow fragment DNA polymerase in the presence of [α³²P]CTP and purified on Qiaquick Spin Columns (Qiagen). Increasing amount (0.25 to 64 pmol) of recombinant c-Fos and recombinant protein (4 pmol) of MafB (wt) or MafB (E269R) and MafB (V277N) mutants were incubated with 0.05 ng of probe in 20 μL of binding reaction buffer (20 mM Tris-HCl (pH 7.5), 100 mM KCl, 0.1 mM EDTA, 5% glycerol, 0.1% Triton X-100, 0.02% BSA, 0.5 mg poly d[I-C]) for 20 min. Complexes formed were resolved on a 6% polyacrylamide (acrylamide/bisacrylamide ratio, 29:1) non-denaturating gel (Bio-Rad) in 0.5% Tris-glycine. Gels were dried and autoradiographed at −80° C.

Co-Immunoprecipitation:

MafB-deficient Maf-DKO macrophages (Aziz et al., 2009) were transduced with indicated combinations of empty, c-Fos and MafB (wt) or MafB (E269R) and MafB (V277N) mutant encoding pMSCV vector using previously described infection protocols (Aziz et al., 2009). Cells were lysed in RIPA buffer (50 mM Tris HCl pH 8, 150 mM NaCl, 1% NP-40, 0.5% sodium deoxycholate, 0.1% SDS, protease inhibitors) and incubated for 30 minutes at 4° C. After clearing by centrifugation, lysate were incubated with the Flag-M2 antibody conjugated to agarose (Sigma, F2426) or with the anti-c-Fos antibody (Santa Cruz, sc-7202), previously coupled with protein A/G. After incubation, pellets were collected by centrifugation and washed four times in washing buffer (50 mM Tris HCl pH 8, 150 mM NaCl, protease inhibitors). Bound proteins were eluted with 62.5 mM Tris HCl, pH 6.8, 2% SDS, 10% glycerol, and 0.002% bromophenol blue. Western blot detection was done by standard methods using anti-c-Fos (Santa Cruz, sc-7202) or anti Flag-HRP conjugated antibody (Sigma, A8592), respectively.

Transient Transfection and Reporter Gene Assay:

HEK293 cells were grown in Dulbecco's modified eagle's medium supplemented with 10% fetal bovine serum in six-well plates to reach 60-80% confluence at the time of transfection. DNA was transfected by calcium phosphate precipitation procedure as previously described (Sieweke et al., 1996). pTK81 luciferase reporter constructs (200 ng) (Promega) containing three multimerized T-MARE(alpha) (TCGACCTCCCTTATGCTGACTCAGCAATTCTCTCGAGCTCCCTTATGCTGACTCAG CA ATTCTCTCGAGCTCCCTTATGCTGACTCAGCAATTCTCGTCGACCC), T-MARE(beta) (TCGACCTCCCCTCCTATGAGTCAGCAATTCTCTCGAGCTCCCCTCCTATGAGTCAG CA ATTCTCTCGAGCTCCCCTCCTATGAGTCAGCAATTCTCGTCGACCC), or T-MARE (y) (TCGACCTCCCTTATGCCTACTAGGCAATTCTCTCGAGCTCCCTTATGCCTACTAGG CAATTCTCTCGAGCTCCCTTATGCCTACTAGGCAATTCTCGTCGACCC) were cotransfected with 200 ng of Rc/CMV (Invitrogen) constructs driving the expression of wt and mutant full-length MafB with or without 200 ng of Rc/CMV construct driving the expression of c-Fos or no transgene (vector control). Assays were performed in duplicate. The transfection efficiency was normalized by assaying for β-galactosidase activity from a cotransfected CVM-LacZ construct, and luciferase activity was analyzed as previously described (Sieweke et al., 1996). For the competitive luciferase assay, Firefly luciferase gene downstream of T-MARE(y) was excised using the BglII and XbaI sites and has been replaced by the Renilla luciferase gene. pTK81-T-MARE(beta)-Firefly and pTK81-T-MARE(y)-Renilla were cotransfected alone, with an Rc/CMV-MafB expression vector or with both MafB and c-Fos expression vector. The transfection efficiency was normalized, by assaying for β-galactosidase activity from a cotransfected CVM-LacZ construct, and luciferase activity was analyzed using a dual-luciferase reporter assay system (Promega, Cat: E1910) according to the manufacturer's recommendations.

Results and Discussion:

Structure Determination of Distinct MafB₂ and MafB/c-Fos DNA Complexes:

To determine the molecular parameters that govern specific binding of the homodimeric MafB₂ and heterodimeric MafB/c-Fos complexes to the T-MARE motif we investigated T-MARE variants with an expected preference for the homodimeric MafB₂ or the heterodimeric MafB/c-Fos assembly. We used the following oligonucleotides: i) a canonical, palindromic T-MARE motif (5′-TGCTGACTCAGCA-3′) with an intact TRE site and two flanking MARE extensions (underlined), referred to as T-MARE(a); ii) an asymmetric oligonucleotide (5′-TGAGTCAGCA-3′) with only one MARE extension (underlined) flanking the central TRE, referred to as T-MARE(f3), to promote MafB/c-Fos heterodimerization; and iii) a third oligonucleotide (5′-TGCCTACTAGGCA-3′) that has two unchanged MARE extensions and an altered TRE core motif (changes in italics), referred to as T-MARE(y), to promote MafB₂ homodimerization.

As shown by an electrophoretic mobility shift assay (EMSA), at an equimolar ratio of MafB and c-Fos, both complexes—homodimeric MafB₂ and heterodimer MafB/c-Fos—are found at comparable concentrations when bound to T-MARE(a). Changing the ratio of the two transcription factors by c-Fos titration resulted in a shift in complex formation: homodimeric MafB₂ in the presence of excess MafB, and heterodimeric MafB/c-Fos in the presence of excess C-Fos. Under identical experimental conditions T-MARE(beta) showed a strong preference for MafB/c-Fos and T-MARE(y) bound only homodimeric MafB₂ with no significant amount of heterodimeric MafB/c-Fos detected.

Using a previously established purification protocol (Textor et al., 2007), we first determined the crystal structure of homodimeric MafB₂ with a 20-bp DNA duplex, encompassing the T-MARE motif, at 2.9 Å resolution. To obtain a pure heterodimeric MafB/c-Fos complex suitable for crystallization we exploited the established binding preference of heterodimeric MafB/c-Fos for T-MARE(beta), allowing the separation of a highly pure MafB/c-Fos/T-MARE(beta) complex and its X-ray structure determination at 2.3 Å resolution.

In the homodimeric MafB₂/T-MARE complex, each MafB protomer symmetrically binds to one of the two T-MARE half-sites. The two MafB molecules show a virtually identical overall conformation, reflected by a root-mean-squares deviation (rmsd) of 1.7 Å for 92 common residues. Based on this structure, we have defined three sequence segments for each MafB polypeptide chain: the first represents the N-terminal EHR, which folds into a small three-helical bundle domain or helix-turn-helix motif (residues 211-237); the second sequence segment represents the BR that binds into the major groove of each T-MARE half-binding site (residues 238-261); the third segment forms the Zip region that establishes the homodimeric MafB assembly by a long, left-handed coiled coil (residues 262-305). The Zip and BR together constitute the basic zipper (bZip) region, which folds into a long, uninterrupted 75-residue helix with more than 20 helical turns.

In contrast to the MafB₂/T-MARE complex, the heterodimer MafB/c-Fos/T-MARE(beta) complex is asymmetric owing to the two different protein ligands (MafB, c-Fos) and the lack of one of the two MARE extensions in the DNA motif. MafB is bound to the remaining T-MARE half-site, whereas c-Fos binds to the opposite TRE half-site without the T-MARE extension. Similarly to MafB, c-Fos comprises a BR (residues 137-160) involved in DNA binding, followed by the Zip segment (residues 161-200) that assembles with the equivalent MafB Zip segment (residues 262-301) into a heterodimeric coiled coil. In contrast to MafB, c-Fos does not comprise an additional EHR.

Recognition of Distinct MARE Variants by MafB₂ Versus MafB/c-Fos:

Comparison of the MafB₂/T-MARE and MafB/c-Fos/T-MARE(beta) complexes revealed remarkable differences in the balance of specific DNA interactions, formed with the central TRE motif and the flanking MARE nucleotides. In the structure of the heterodimeric MafB/c-Fos/T-MARE(beta) complex the observed protein—DNA interactions are highly asymmetric and are caused by mostly unrelated interactions from the two protein ligands. Most of the MafB-mediated interactions with the DNA half-site, which contain the remaining T-MARE extension, are identical to those found in the MafB₂/T-MARE complex. An exception is R256 from MafB that provides an additional base-specific interaction via a bidentate hydrogen bond pattern with guanine in the central position 0 of the heterodimeric MafB/c-Fos/T-MARE(beta) complex. By contrast, the equivalent c-Fos residue (R155) is only involved in interactions with the DNA phosphate backbone. In addition, a total of six residues of the BR from c-Fos are involved in T-MARE(beta) interactions that are mostly restricted to the central TRE of T-MARE(beta). Only one c-Fos residue, N147, is involved in base-specific interactions by hydrogen bonds to C(+2) and T(−3), which are located on opposite strands within the same TRE half-site. N147 from c-Fos is structurally equivalent to N248 from MafB, which is one of two MafB key residues for base-specific interactions with T-MARE. Remarkably, a spatial difference of the asparagine side chain by about 2 Å is sufficient to allow different base-specific interactions either with the central TRE (mediated by c-Fos) or with the extended T-MARE (mediated by MafB). The other c-Fos residue, R143, which has a conserved structural MafB equivalent involved in base-specific interactions (R244), does not bind to any T-MARE(beta) base.

In contrast to the MafB/c-Fos/T-MARE(beta) complex, the protein—DNA interactions observed in the homodimeric MafB₂/T-MARE complex are almost identical in the two MafB protomers, reflecting the symmetric nature of the overall complex. Whereas interactions with the DNA phosphate backbone are distributed over the complete T-MARE, base-specific interactions by residues R244 and N248 are restricted to the G/C positions+/−4 and +/−5 of the two T-MARE-specific, extended three-base elements. Interestingly, no residues from the EHR are involved in any side-chain-mediated T-MARE interactions, except V221 from the N-terminus of the second helix of the EHR.

A comparative analysis of the protein interactions with the DNA phosphate backbone of the two complexes revealed that the majority of the residues contributing to these interactions are not conserved in MafB and c-Fos. Two MafB residues, 8243 and Y251, which contribute to DNA-backbone recognition of the extended T-MARE base triplet by hydrogen bonds, are substituted with small hydrophobic residues in c-Fos (1142, A150). Conversely, a c-Fos residue (R159) that binds to the phosphate group of one of the central TRE bases is replaced in MafB with a small hydrophobic amino acid (V260) that does not have the ability to bind to DNA, thus shifting the overall balance of MafB-mediated DNA phosphate interactions towards the extended T-MARE base triplet. However, as the key MafB residues responsible for base-specific interactions are conserved in c-Fos (MafB/c-Fos, R244/R143 and N248/N147), our structural data indicate that the ability to form a different set of additional phosphate-mediated DNA backbone interactions, namely mediated by MafB-specific residues 8243 and Y251, is an important parameter for the different preferred DNA-recognition sequences by c-Fos and MafB, represented by TRE and the extended T-MARE half-sites, respectively. Together these structural data reveal the molecular basis of distinct DNA-binding site preferences for MafB₂ homodimers and MafB/c-Fos heterodimers.

Molecular Parameters Permitting Facultative MafB₂ and MafB/c-Fos Assembly:

We next analyzed the structures of the MafB₂ and MafB/c-Fos complexes to identify the molecular parameters that determine homo-versus heterodimer formation. The overall coiled-coil arrangement in MafB₂ and MafB/c-Fos extends over six complete heptad repeats, generating extensive interface surfaces (1275 Å² in MafB₂, 1058 Å² in MafB/c-Fos). Most of the coiled-coil interactions are found within the four central heptad repeats II-V of both complexes whereas the flanking repeats I and VI are more loosely arranged. In both protein assemblies, most of the specificity-determining interactions are found in repeats II, III and V, whereas repeat IV is dominated by hydrophobic knob-into-holes interactions.

Repeat position d in the first four repeats of MafB is a highly conserved leucine, which is the most common amino acid in this position in canonical coiled coils (Miller, 2009). However in repeat V, it is occupied by an unusual aromatic amino acid (Y294). In the homodimeric MafB₂ complex, the phenyl side chains of the same residue from the two MafB helices form a stacked, parallel-layered interaction. In each of the MafB repeats I, II and V, position a is represented by positively charged residues, which is rare in other bZip coiled coils. Two of these residues contribute to the two salt-bridge pairs of the MafB₂ homodimer, along with negatively charged residues from neighboring g positions: E269-K270 (heptad II) and E290-R291 (heptad V).

Surprisingly and in marked contrast to most other coiled-coil arrangements in bZip transcription factors (Miller, 2009; Vinson et al., 2006), there are no specific interactions between any residues from heptad positions e and g. The conclusion on distinct selectivity rules is supported by a comparison of the MafB₂/T-MARE complex with a recent structure of protein/DNA complex of MafG (Kurokawa et al., 2009), which is member of the distantly related family of small Maf transcription factors that shares the same domain structure responsible for coiled coil-mediated protein—protein assembly and DNA binding but lacks a transactivation domain (Eychene et al., 2008). Although the interface positions (a, d, e, g) of the coiled-coil region from MafB and MafG share 50% sequence identity (12/24 residues from six heptads), only one specific interaction—E269-K270 in MafB—is structurally conserved, whereas all further coiled-coil interaction pairs are either specific for MafG or MafB. Another structure of MafA (Lu et al., 2012), a member of the long Maf family, was not included into this comparison since many amino acid side chains in the leucine zipper region were only partially built.

In the c-Fos heptad repeat sequence all d positions are represented by leucine residues. Positions a of repeats III and V, similar to our observations in MafB, are positively charged residues. K176 (c-Fos) from the a position of repeat III forms an additional hydrogen bond with Q276 (MafB) from the preceding g position, which is not observed in the MafB₂ coiled coil. However, in homodimeric MafB₂ such an interaction is not possible as heptad III is the only one with a small hydrophobic residue (V277) in the respective a position.

However, as each of the a positions in repeat V of MafB and c-Fos contains a positively charged residue and the g positions from the preceding heptad are represented by a glutamate, a pair of salt bridges—E290 (MafB)-K190 (c-Fos) and R291 (MafB)-E189 (c-Fos)—is formed that is in a virtually identical position to the symmetric pair of salt bridges E290-R291 in the MafB₂ coiled coil. For the other symmetric salt-bridge pair in the homodimeric MafB₂ assembly, E269-K270, only one of the two equivalent c-Fos residues is a charged residue (E168), and so only one salt bridge can be formed in the heterodimeric MafB/c-Fos coiled coil: E168 (c-Fos)-K270 (MafB).

Thus, the total number of specific salt-bridge interactions from residues of heptad positions a and g in the heterodimeric MafB/c-Fos complex is four, like in the homodimeric MafB₂ complex. Of these, three are structurally conserved in both bZip assemblies. Like in the homodimeric MafB₂ complex, no single, specific interaction between positions e and g is found in the heterodimeric MafB/c-Fos complex. In addition, an unusual specific interaction is formed between Y294 (MafB) from position d in heptad V and E194 (c-Fos) from position e in heptad V.

A comparison of the specific heptad interactions found in the c-Fos/MafB/T-MARE(beta) and in the c-Fos/c-Jun/TRE complexes (Glover and Harrison, 1995) directly demonstrates a significantly larger number of interactions by heptad positions a-g and e-g in the latter assembly. In contrast to bZip complexes with extended MARE-type DNA-recognition specificities (MafB, MafG), in the c-Fos/c-Jun complex there are no predictable specific interactions missing (Grigoryan et al., 2009; Newman and Keating, 2003). None of the five identified heptad interactions from the e-g positions in the c-Fos/c-Jun complex is found in the new heterodimeric c-Fos/MafB complex. Since the DNA-binding preferences for c-Fos/c-Jun (TRE) and c-Fos/MafB are different, proper heterodimeric bZip assembly involving c-Fos via the underlying specific coiled-coil protein-protein interactions permits selective recognition of bZip-specific cognate DNA motifs.

Designed MafB Mutants with Altered Dimerization Properties and DNA-Binding Profile:

We used this precise knowledge about the molecular parameters that support distinct MafB₂ and MafB/c-Fos dimerization to design structure-based MafB mutants with altered dimerization preferences. To engineer a MafB version that is expected to favor heterodimerization with c-Fos, at the expense of the ability for homodimerization, we mutated E269 from the g position in heptad II into an arginine. We predicted that this MafB version would generate a repulsive interaction between K270 and the additional positive charge introduced in residue 269, and therefore would lose the ability to form the specific homodimeric salt-bridge interaction observed in the MafB₂ complex. As the residue equivalent to K270 in MafB is replaced by a threonine in c-Fos, we reasoned that this mutation should have no negative effect on heterodimeric MafB/c-Fos complex formation. By contrast, we predicted that the E269R mutant could engage in an attractive interaction with E173 of c-Fos in the MafB/c-Fos heterodimer. Our prediction that the MafB (E269R) mutant would favor heterodimeric MafB/c-Fos assembly was confirmed by co-immunoprecipitation data showing an increased ability of the mutant to interact with c-Fos. Furthermore, EMSA analysis revealed that even at a sixteen-fold excess of MafB over c-Fos, this mutant could still mediate heterodimer formation and, at equimolar ratios, resulted in almost exclusive MafB/c-Fos complex formation with only trace amounts of the homodimeric MafB₂. This is in contrast to the wild-type (wt) protein that does not form heterodimers in the presence of excess MafB and forms both types of complexes at equimolar ratios.

To engineer a MafB version with a preference for homodimerization, we mimicked known coiled coil bZip transcription factor assemblies that contain several glutamine/asparagine pairs with mixed donor/acceptor abilities of the two residues in neighboring g and a heptad positions (Schumacher et al., 2000). This type of residue pair allows the formation of a unique, stable assembly layer via a Q-N-N-Q hydrogen bond wire, maximizing the number of possible lateral hydrogen bonds per heptad repeat. Such an interaction is observed neither in the homodimeric MafB₂ assembly nor in the heterodimeric MafB/c-Fos complex. By mutating V277 of MafB into an asparagine we designed such a motif allowing the formation of a Q276-N277-N277-Q276 hydrogen bond wire in the homodimeric MafB₂ but not in the heterodimeric MafB/c-Fos complex. Consistent with a stabilization of homodimeric interactions, we expected this mutant to prefer homodimer rather than heterodimer formation under conditions in which both interactions are equally possible. This prediction was confirmed by co-immunoprecipitation experiments, in which MafB (V277N) showed stronger homodimerization than MafB (wt) protein and EMSA on a T-MARE, in which MafB (V277N) almost exclusively formed homodimers and no MafB/c-Fos complexes even at a 20-fold excess of c-Fos. Taken together, this study shows that, by making use of structural leucine zipper interaction data, it is possible to alter the balance of homo- and heterodimeric MafB complexes in the presence of the same T-MARE sequence by introducing additional targeted attractive or repulsive interactions.

Based on our findings of altered homo- and heterodimeric assembly properties, we further investigated to what extent these MafB mutants could select for different T-MARE motifs. The MafB (E269R) variant indeed showed strong differential activity on T-MARE(gamma)- and T-MARE(beta)-binding sites. Compared with MafB (wt), it exhibited detectable binding to homodimer-promoting T-MARE(gamma) only in the presence of excessive amounts of MafB and exclusive heterodimer formation on heterodimer-promoting T-MARE(beta), even at residual c-Fos concentrations. Consistent with this, MafB (E269R) showed a five-fold higher transactivation of a synthetic T-MARE(beta) reporter in the presence of c-Fos than the wt protein and a reduced activity on a T-MARE(gamma) reporter. To test whether these observations would lead to a selective and preferential activation of T-MARE(beta)-containing promoters when both MARE variant binding sites are available, we established a competitive transactivation assay with a T-MARE(gamma) promoter-driven renilla luciferase reporter and a T-MARE(beta) promoter-driven firefly luciferase reporter, which can be individually quantified in the same cell extract. When transfecting both reporters together with either MafB (wt) or MafB (E269R) we observed that MafB (wt) showed a strong preference for T-MARE(gamma) promoter activation even in the presence of c-Fos, whereas MafB (E269R) strongly selected for T-MARE(beta) promoter activation, particularly in the presence of c-Fos. Together these data indicate that a single amino acid variation in MafB can induce a strong shift from activating T-MARE(gamma)- to T-MARE(beta)-containing promoters and thus select both negatively against MafB activity on T-MARE(gamma), and positively for MafB activity on T-MARE(beta) sites.

However, when we investigated the MafB (V277N) mutant, we did not observe a comparable effect on preferential T-MARE(y)-binding site selection, possibly because the T-MARE(y) variant already strongly favors homodimer binding that may be difficult to further enhance. The binding of this MafB variant to the heterodimer-promoting T-MARE(beta) site was also unchanged. This finding was expected, as unlike the MafB (E269R) mutant, the MafB (V277N) mutant does not generate any predicted repulsive interactions in the MafB/c-Fos heterodimeric complex. This observation was further confirmed in transactivation assays, in which the MafB (V277N) mutant showed no significant difference to the wt protein on synthetic T-MARE(y) and T-MARE(beta) reporters.

### CONCLUSION

During recent years, regulated systems for gene transcription have been increasingly employed to alter genetic programs and associated functional readouts, such as signaling and metabolic pathways (Kiel et al., 2010; Lim, 2010; Tigges and Fussenegger, 2009). In this contribution, we show for the first time that, using two structures of the same bZip transcription factor MafB either assembled as a homodimer or as a heterodimer with another bZip transcription factor c-Fos, it is possible to change the balance of MafB₂ homodimer and MafB/c-Fos heterodimer formation on the same T-MARE sequence by targeted mutations in the respective leucine zippers. We also show that it is possible to change the binding preference for different T-MARE variants, as shown for the MafB (E269R) mutant. In general terms, our findings thus could provide new tools to control specific gene expression by selectively activating homo- or heterodimer-specific binding repertoires and signaling pathways. As the basic principles of coiled-coil protein/protein interactions are common to all bZip transcription factors, our procedures could be applicable to other members of this transcription factor family, to the extent that the DNA-recognition motifs of partner bZip transcriptions factors differ. Engineered MafB dimerization may ultimately enable the precise understanding and controlled manipulation of distinct activity states and binding repertoires of MafB, in particular in macrophages, with therapeutic potential in infectious disease, inflammatory or autoimmune disorders, regeneration and tumor biology.

### REFERENCES

Throughout this application, various references describe the state of the art to which this invention pertains. The disclosures of these references are hereby incorporated by reference into the present disclosure.
Adams, P. D., Afonine, P. V., Bunkoczi, G., Chen, V. B., Echols, N., Headd, J. J., Hung, L. W., Jain, S., Kapral, G. J., Grosse Kunstleve, R. W., et al. (2011). The Phenix software for automated determination of macromolecular structures. Methods 55, 94-106.
Amit, I., Garber, M., Chevrier, N., Leite, A. P., Donner, Y., Eisenhaure, T., Guttman, M., Grenier, J. K., Li, W., Zuk, O., et al. (2009). Unbiased reconstruction of a mammalian transcriptional network mediating pathogen responses. Science 326, 257-263.
Aziz, A., Soucie, E., Sarrazin, S., and Sieweke, M. H. (2009). MafB/c-Maf deficiency enables self-renewal of differentiated functional macrophages. Science 326, 867-871.
Bricogne, G., Vonrhein, C., Flensburg, C., Schiltz, M., and Paciorek, W. (2003). Generation, representation and flow of phase information in structure determination: recent developments in and around SHARP 2.0. Acta Crystallogr D Biol Crystallogr 59, 2023-2030.
Collaborative Computational Project Number 4 (1994). Collaborative Computational Project, Number 4. Acta Crystallogr D Biol Crystallogr 50, 760-763.
Emsley, P., and Cowtan, K. (2004). Coot: model-building tools for molecular graphics. Acta Crystallogr D Biol Crystallogr 60, 2126-2132.
Eychene, A., Rocques, N., and Pouponnot, C. (2008). A new MAFia in cancer. Nat Rev Cancer 8, 683-693.
Glover, J. N., and Harrison, S. C. (1995). Crystal structure of the heterodimeric bZIP transcription factor c-Fos-c-Jun bound to DNA. Nature 373, 257-261.
Grigoryan, G., and Keating, A. E. (2008). Structural specificity in coiled-coil interactions. Curr Opin Struct Biol 18, 477-483.
Grigoryan, G., Reinke, A. W., and Keating, A. E. (2009). Design of protein-interaction specificity gives selective bZIP-binding peptides. Nature 458, 859-864.
Igarashi, K., Kataoka, K., Itoh, K., Hayashi, N., Nishizawa, M., and Yamamoto, M. (1994). Regulation of transcription by dimerization of erythroid factor NF-E2 p45 with small Maf proteins. Nature 367, 568-572.
Introna, M., Hamilton, T. A., Kaufman, R. E., Adams, D. O., and Bast, R. C., Jr. (1986). Treatment of murine peritoneal macrophages with bacterial lipopolysaccharide alters expression of c-fos and c-myc oncogenes. J Immunol 137, 2711-2715.
Kabsch, W. (2010). Xds. Acta Crystallogr D Biol Crystallogr 66, 125-132.
Kerppola, T. K., and Curran, T. (1994). A conserved region adjacent to the basic domain is required for recognition of an extended DNA binding site by Maf/Nrl family proteins. Oncogene 9, 3149-3158.
Kiel, C., Yus, E., and Serrano, L. (2010). Engineering signal transduction pathways. Cell 140, 33-47.
Koga, K., Takaesu, G., Yoshida, R., Nakaya, M., Kobayashi, T., Kinjyo, I., and Yoshimura, A. (2009). Cyclic adenosine monophosphate suppresses the transcription of proinflammatory cytokines via the phosphorylated c-Fos protein. Immunity 30, 372-383.
Kurokawa, H., Motohashi, H., Sueno, S., Kimura, M., Takagawa, H., Kanno, Y., Yamamoto, M., and Tanaka, T. (2009). Structural basis of alternative DNA recognition by Maf transcription factors. Mol Cell Biol 129, 6232-6244.
Kusunoki, H., Motohashi, H., Katsuoka, F., Morohashi, A., Yamamoto, M., and Tanaka, T. (2002). Solution structure of the DNA-binding domain of MafG. Nat Struct Biol 9, 252-256.
Laskowski, R. A., MacArthur, M. W., and Thornton, J. M. (1998). Validation of protein models derived from experiment. Curr Opin Struct Biol 8, 631-639.
Laskowski, R. A., Moss, D. S., and Thornton, J. M. (1993). Main-chain bond lengths and bond angles in protein structures. J Mol Biol 231, 1049-1067.
Leslie, A. G. W., and Powell, H. R. (2007). Processing diffraction data with MOSFLM. In Evolving Methods for Macromolecular Crystallography (Dordrecht, The Netherlands: Springer), pp. 41-51.
Lim, W. A. (2010). Designing customized cell signalling circuits. Nat Rev Mol Cell Biol 11, 393-403.
Lu, X., Guanga, G. P., Wan, C., and Rose, R. B. (2012). A Novel DNA Binding Mechanism for maf Basic Region-Leucine Zipper Factors Inferred from a MafA-DNA Complex Structure and Binding Specificities. Biochemistry.
McCoy, A. J. (2007). Solving structures of protein complexes by molecular replacement with Phaser. Acta Crystallogr D Biol Crystallogr 63, 32-41.
Miller, M. (2009). The importance of being flexible: the case of basic region leucine zipper transcriptional regulators. Curr Protein Pept Sci 10, 244-269.
Miyatake, H., Hasegawa, T., and Yamano, A. (2006). New methods to prepare iodinated derivatives by vaporizing iodine labelling (VIL) and hydrogen peroxide VIL (HYPER-VIL). Acta Crystallogr D Biol Crystallogr 62, 280-289.
Murshudov, G. N., Vagin, A. A., and Dodson, E. J. (1997). Refinement of macromolecular structures by the maximum-likelihood method. Acta Crystallogr D Biol Crystallogr 53, 240-255.
Newman, J. R., and Keating, A. E. (2003). Comprehensive identification of human bZIP interactions with coiled-coil arrays. Science 300, 2097-2101.
Ogata, K., Sato, K., and Tahirov, T. H. (2003). Eukaryotic transcriptional regulatory complexes: cooperativity from near and afar. Curr Opin Struct Biol 13, 40-48.
Panne, D. (2008). The enhanceosome. Curr Opin Struct Biol 18, 236-242.
Pulendran, B., Tang, H., and Manicassamy, S. (2010). Programming dendritic cells to induce T(H)2 and tolerogenic responses. Nat Immunol 11, 647-655.
Remenyi, A., Scholer, H. R., and Wilmanns, M. (2004). Combinatorial control of gene expression. Nat Struct Mol Biol 11, 812-815.
Sarrazin, S., Mossadegh-Keller, N., Fukao, T., Aziz, A., Mourcin, F., Vanhille, L., Kelly Modis, L., Kastner, P., Chan, S., Duprez, E., et al. (2009). MafB restricts M-CSF-dependent myeloid commitment divisions of hematopoietic stem cells. Cell 138, 300-313.
Schneider, T. R., and Sheldrick, G. M. (2002). Substructure solution with SHELXD. Acta Crystallogr D Biol Crystallogr 58, 1772-1779.
Schumacher, M. A., Goodman, R. H., and Brennan, R. G. (2000). The structure of a CREB bZIP.somatostatin CRE complex reveals the basis for selective dimerization and divalent cation-enhanced DNA binding. J Biol Chem 275, 35242-35247.
Sieweke, M. H., and Graf, T. (1998). A transcription factor party during blood cell differentiation. Curr Opin Genet Dev 8, 545-551.
Sieweke, M. H., Tekotte, H., Frampton, J., and Graf, T. (1996). MafB is an interaction partner and repressor of Ets-1 that inhibits erythroid differentiation. Cell 85, 49-60.
Textor, L. C., Wilmanns, M., and Holton, S. J. (2007). Expression, purification, crystallization and preliminary crystallographic analysis of the mouse transcription factor MafB in complex with its DNA-recognition motif Cmare. Acta Crystallogr Sect F Struct Biol Cryst Commun 63, 657-661.
Tigges, M., and Fussenegger, M. (2009). Recent advances in mammalian synthetic biology-design of synthetic transgene control networks. Curr Opin Biotechnol 20, 449-460.
Vinson, C., Acharya, A., and Taparowsky, E. J. (2006). Deciphering B-ZIP transcription factor interactions in vitro and in vivo. Biochim Biophys Acta 1759, 4-12.
Yamamoto, T., Kyo, M., Kamiya, T., Tanaka, T., Engel, J. D., Motohashi, H., and Yamamoto, M. (2006). Predictive base substitution rules that determine the binding and transcriptional specificity of Maf recognition elements. Genes Cells 11, 575-591.

1. A MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid.

2. The MafB mutant polypeptide of claim 1 wherein the basic amino acid is selected from the group consisting of arginine, lysine or histidine.

3. (canceled)

4. The MafB mutant polypeptide of claim 1 wherein the polar amino acid is selected from the group consisting of serine (Ser), threonine (Thr), cysteine (Cys), asparagine (Asn), glutamine (Gln), and tyrosine (Tyr).

5. The MafB mutant polypeptide of claim 1 which is or comprises a sequence having at least 90% amino acid identity with SEQ ID NO: 1 or 2 providing that the glutamic acid residue at position 269 has been deleted or substituted with a basic amino acid and/or the valine residue at position 277 has been deleted or substituted with a polar amino acid.

6. The MafB mutant polypeptide of claim 1 which is or comprises a sequence having 90; 91; 92; 93; 94; 95; 96; 97; 98 or 99% amino acid identity with SEQ ID NO: 1 or 2 providing that the glutamic acid residue at position 269 has been deleted or substituted with a basic amino acid or the valine residue at position 277 has been deleted or substituted with a polar amino acid.

7. A fusion protein comprising a MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid, wherein the MafB mutant polypeptide is fused to a heterologous polypeptide.

8. An isolated nucleic acid molecule that encodes
i) MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid,
or
ii) a fusion protein comprising the MafB mutant polypeptide fused to a heterologous polypeptide.

9. A recombinant vector comprising a nucleic acid molecule that encodes
i) a MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid,
or
ii) a fusion protein comprising the MafB mutant polypeptide fused to a heterologous polypeptide.

10. A host cell into which a recombinant expression vector has been introduced, the recombinant vector comprising a nucleic acid molecule that encodes
i) a MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid,
or
ii) a fusion protein comprising the MafB mutant polypeptide fused to a heterologous polypeptide.

11. The host cell of claim 10 which is a monocyte or a macrophage.

12. The host cell of claim 11 which is a self-renewing monocyte or macrophage.

13. A method of producing a MafB mutant polypeptide comprising the steps of: (i) culturing a transduced host cell according to the invention into which a recombinant expression vector has been introduced under conditions suitable to allow expression of said MafB mutant polypeptide; wherein the recombinant expression vector encodes the MafB mutant polypeptide or a fusion protein comprising the MafB mutant polypeptide and (ii) recovering the expressed MafB mutant polypeptide,
wherein the MafB mutant polypeptide comprises an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid.

14. A non-human transgenic animal in which exogenous nucleic acid sequences encoding a MafB mutant polypeptide have been introduced into a genome of the non-human transgenic animal,
wherein the MafB mutant polypeptide comprises an amino acid sequence as set forth in SEQ ID NO: 1 or 2, or a function conservative variant thereof, and wherein a glutamic acid residue at position 269 has been deleted or substituted with a basic amino acid, and/or wherein a valine residue at position 277 has been deleted or substituted with a polar amino acid.

15. An antibody specific for a MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid.

16. (canceled)

17. A method for screening a plurality of candidate compounds comprising the steps of
(a) testing each of the plurality of candidate compounds for its ability to inhibit activity of a MafB/MafB homodimeric or MafB/c-Fos heterodimeric complex in a host cell, wherein the host cell comprises a recombinant vector comprising a nucleic acid molecule that encodes i) a MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid, or ii) a fusion protein comprising the MafB mutant polypeptide fused to a heterologous polypeptide; and
(b) and positively selecting the candidate compounds capable of inhibiting said activity.

18. A pharmaceutical composition comprising
a host cell comprising a recombinant vector comprising a nucleic acid molecule that encodes i) a MafB mutant polypeptide comprising an amino acid sequence as set forth in SEQ ID NO: 1 or 2, or a function conservative variant thereof, wherein a glutamic acid residue at position 269 has been deleted or substituted with a basic amino acid, and/or wherein a valine residue at position 277 has been deleted or substituted with a polar amino acid, or ii) a fusion protein comprising the MafB mutant polypeptide fused to a heterologous polypeptide; and
a pharmaceutically acceptable carrier.


SEQUENCE LISTING (13 sequences)

SEQ ID NO: 1 - protein, 322 aa, Homo sapiens
MAAELSMGPELPTSPLAMEYVNDFDLLKFDVKKEPLGRAERPGRPCTRLQPAGSVSSTPLTPCSSVPSSPSFSPTEQKTHLEDLYWMASNYQQMNPEALNLTPEDAVEALIGSHPVPQPLQSFDSFRGAHHHHHHHHPHPHHAYPGAGVAHDELGPHAHPHHHHHHQASPPPSSAASPAQQLPTSHPGPGPHATASATAAGGNGSVEDRFSDDQLVSMSVRELNRHLRGFTKDEVIRLKQKRRTLKNRGYAQSCRYKRVQQKHHLENEKTQLIQQVEQLKQEVSRLARERDAYKVKCEKLANSGFREAGSTSDSPSSPEFFL

SEQ ID NO: 2 - protein, 323 aa, Mus musculus
MAAELSMGQELPTSPLAMEYVNDFDLLKFDVKKEPLGRAERPGRPCTRLQPAGSVSSTPLSTPCSSVPSSPSFSPTEPKTHLEDLYWMASNYQQMNPEALNLTPEDAVEALIGSHPVPQPLQSFDGFRSAHHHHHHHHPHPHHGYPGAGVTHDDLGQHAHPHHHHHHQASPPPSSAASPAQQLPTSHPGPGPHATAAATAAGGNGSVEDRFSDDQLVSMSVRELNRHLRGFTKDEVIRLKQKRRTLKNRGYAQSCRYKRVQQKHHLENEKTQLIQQVEQLKQEVSRLARERDAYKVKCEKLANSGFREAGSTSDSPSSPEFFL

SEQ ID NO: 3 - DNA, 20 bp, Artificial
  Synthetic oligonucleotide primer
taattgctgactcagcaatt

SEQ ID NO: 4 - DNA, 20 bp, Artificial
  Synthetic oligonucleotide primer
taattgctgagtcagcaatt

SEQ ID NO: 5 - DNA, 29 bp, Artificial
  Synthetic T-Mare(a) oligonucleotide
tcgtcccttatgctgactcagcaattctc

SEQ ID NO: 6 - DNA, 29 bp, Artificial
  Synthetic T-MARE(b) oligonucleotide
tcgtcccctcctatgagtcagcaattctc

SEQ ID NO: 7 - DNA, 29 bp, Artificial
  Synthetic T-MARE(y) oligonucleotide
tcgtcccttatgcctactaggcaattctc

SEQ ID NO: 8 - DNA, 104 bp, Artificial
  Synthetic multimerized T-MARE(a) oligonucleotide
tcgacctcccttatgctgactcagcaattctctcgagctcccttatgctgactcagcaattctctcgagctcccttatgctgactcagcaattctcgtcgaccc

SEQ ID NO: 9 - DNA, 104 bp, Artificial
  Synthetic multimerized T-MARE-(b) oligonucleotide
tcgacctcccctcctatgagtcagcaattctctcgagctcccctcctatgagtcagcaattctctcgagctcccctcctatgagtcagcaattctcgtcgaccc

SEQ ID NO: 10 - DNA, 104 bp, Artificial
  Synthetic multimerized T-MARE(y) oligonucleotide
tcgacctcccttatgcctactaggcaattctctcgagctcccttatgcctactaggcaattctctcgagctcccttatgcctactaggcaattctcgtcgaccc

SEQ ID NO: 11 - DNA, 13 bp, Artificial
  Synthetic T-MARE motif
tgctgactcagca

SEQ ID NO: 12 - DNA, 10 bp, Artificial
  Synthetic T-MARE asymetric motif
tgagtcagca

SEQ ID NO: 13 - DNA, 13 bp, Artificial
  Synthetic T-MARE(y) motif
tgcctactaggca